(19) United States
(12) Patent Application Publication
(10) Pub. No.: US 2024/0172979 A1
(43) Pub. Date: May 30, 2024

(54) Concentration State Evaluation Method, Non-Transitory Computer-Readable Storage Medium Storing Concentration State Evaluation Program, And Concentration State Evaluation Device

(71) Applicant: SEIKO EPSON CORPORATION, Tokyo (JP)

(72) Inventor: Ikuo HAYAISHI, Asahi (JP)

(21) Appl. No.: 18/517,020

(22) Filed: Nov. 22, 2023

(30) Foreign Application Priority Data
Nov. 24, 2022 (JP) 2022-187499

(51) Int. Cl. A61B 5/16; A61B 5/00; A61B 5/024; A61B 5/352

(52) U.S. Cl. CPC A61B 5/165; A61B 5/02416; A61B 5/352; A61B 5/7271

(57) ABSTRACT

A computer executes: a first measurement step of measuring an interbeat interval during each period of a resting period and a specified working period, based on reference pulse wave information of a user; a second measurement step of measuring an interbeat interval during the desk work period, based on target pulse wave information of the user; an index value calculation step of calculating rMSSD as an index value of a concentration state, based on the interbeat interval measured in the first measurement step or the second measurement step, and finding an index value group corresponding to each period of the resting period, the specified working period, and the desk work period; and an evaluation step of analyzing the index value group corresponding to the desk work period with reference to each of the index value group corresponding to the resting period and the index value group corresponding to the specified working period, and thus evaluating the concentration state of the user during the desk work period.

The present application is based on, and claims priority from JP Application Serial Number 2022-187499, filed Nov. 24, 2022, the disclosure of which is hereby incorporated by reference herein in its entirety.

### BACKGROUND

### 1. Technical Field

The present disclosure relates to a concentration state evaluation method, a non-transitory computer-readable storage medium storing a concentration state evaluation program, and a concentration state evaluation device.

### 2. Related Art

According to the related art, a method of acquiring biometric information of a user and evaluating the physical state of the user based on the biometric information is known. JP-A-2016-190025 is an example of the related art.

The method described in JP-A-2016-190025 detects the pulse rate of the user, performs statistical analysis based on the number of pulses and the velocity of pulse wave propagation, and thus determines the stress level of the user.

However, biometric information such as pulse waves of a user varies from one individual to another. Therefore, with the method using statistical analysis as described in JP-A-2016-190025, it is difficult to accurately evaluate the physical states of individual users.

Also, in view of the recent demand for higher operational efficiency of desk work, the present applicant considers evaluating the concentration state of a user during desk work as the physical state of the user.

### SUMMARY

According to a first aspect of the present disclosure, a concentration state evaluation method for a computer to evaluate a concentration state of a user is provided. The concentration state evaluation method causes the computer to execute: a first measurement step of acquiring reference pulse wave information that represents a pulse wave detected by a pulse wave sensor worn by the user and that represents a pulse wave during each period of a resting period and a specified working period of the user, and measuring an interbeat interval during each period of the resting period and the specified working period, based on the reference pulse wave information; a second measurement step of acquiring target pulse wave information that represents a pulse wave detected by the pulse wave sensor and that represents a pulse wave during an arbitrary desk work period of the user, and measuring an interbeat interval during the desk work period, based on the target pulse wave information; an index value calculation step of calculating rMSSD, which is a root mean square of a difference between adjacent interbeat intervals for each predetermined time, as an index value of the concentration state, based on the interbeat interval measured in the first measurement step or the second measurement step, and finding an index value group corresponding to each period of the resting period, the specified working period, and the desk work period; and an evaluation step of analyzing the index value group corresponding to the desk work period with reference to each of the index value group corresponding to the resting period and the index value group corresponding to the specified working period, and thus evaluating the concentration state of the user during the desk work period.

In the concentration state evaluation method according to the first aspect, the evaluation step may include: a threshold decision step of deciding a resting threshold based on the index value group corresponding to the resting period, and deciding a working threshold based on the index value group corresponding to the specified working period; and a level placement step of placing the index value group corresponding to the desk work period at a plurality of levels of concentration, based on the resting threshold and the working threshold.

In the concentration state evaluation method according to the first aspect, the evaluation step may further include: a normalization step of dividing an analysis range of the index value group corresponding to the desk work period into a plurality of sections, based on the working threshold and the resting threshold, and normalizing, for each of the sections, the index value group corresponding to the desk work period to a range corresponding to the section, before the level placement step.

In the concentration state evaluation method according to the first aspect, the specified working period may include a plurality of working periods with different types of work from each other. The threshold decision step may decide a plurality of thresholds corresponding to the working periods, respectively, as the working threshold.

In the concentration state evaluation method according to the first aspect, the second measurement step may measure each of an interbeat interval and a pulse rate during the desk work period, based on the target pulse wave information. The evaluation step may further include a data sorting step of specifying a pulse rate abnormality period in which the pulse rate is out of a predetermined range during the desk work period, and eliminating the index value corresponding to the pulse rate abnormality period from the index value group corresponding to the desk work period, before the level placement step.

In the concentration state evaluation method according to the first aspect, the evaluation step may further include an analysis step of generating result information representing a change with time across the plurality of levels of concentration during the desk work period, after the level placement step.

In the concentration state evaluation method according to the first aspect, the evaluation step may further include an analysis step of generating result information representing a time proportion of each of the levels of concentration during the desk work period, after the level placement step.

According to a second aspect of the present disclosure, a non-transitory computer-readable storage medium storing a concentration state evaluation program is provided. The concentration state evaluation program is readable and executable by a computer and causes the computer to execute the concentration state evaluation method according to the first aspect.

According to a third aspect of the present disclosure, a concentration state evaluation device includes: a measurement unit that acquires pulse wave information representing a pulse wave detected by a pulse wave sensor worn by a user and representing a pulse wave during each period of a resting period, a specified working period, and an arbitrary desk work period of the user, and that measures an interbeat interval during each of the periods, based on the pulse wave information; an index value calculation unit that calculates rMSSD, which is a root mean square of a difference between adjacent interbeat intervals for each predetermined time, as an index value of a concentration state, based on the interbeat interval measured by the measurement unit, and that finds an index value group corresponding to each period of the resting period, the specified working period, and the desk work period; and an evaluation unit that analyzes the index value group corresponding to the desk work period with reference to each of the index value group corresponding to the resting period and the index value group corresponding to the specified working period, and thus evaluates the concentration state of the user during the desk work period.

### BRIEF DESCRIPTION OF THE DRAWINGS

FIG. 1 is a block diagram showing a schematic configuration of a concentration state evaluation device according to an embodiment of the present disclosure.

FIG. 2 is a block diagram showing the configuration of an evaluation unit in the concentration state evaluation device according to the embodiment.

FIG. 3 is a flowchart for explaining a first stage of a concentration state evaluation method according to the embodiment.

FIG. 4 is a flowchart for explaining a second stage of the concentration state evaluation method according to the embodiment.

FIG. 5 is a graph illustrating the transition of an index value acquired by the processing shown in the flowchart of FIG. 3.

FIG. 6 is a graph for explaining a normalization step in the concentration state evaluation method according to the embodiment.

FIG. 7 is a graph illustrating the transition of the degree of concentration and the level of concentration during a desk work period.

FIG. 8 is a pie chart illustrating the time proportion of each level of concentration during the desk work period.

### DESCRIPTION OF EMBODIMENTS

An embodiment of the present disclosure will now be described.

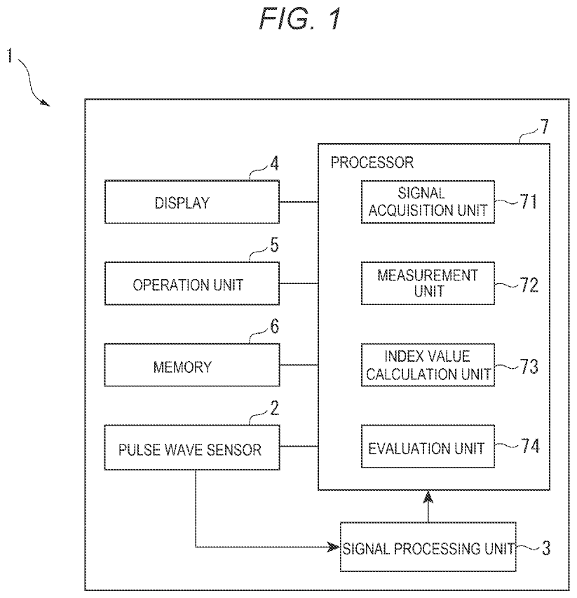

FIG. 1 is a block diagram showing the configuration of a concentration state evaluation device 1 according to this embodiment. The concentration state evaluation device 1 according to this embodiment is configured as a wearable device configured to be wearable on a human body, and evaluates the concentration state of a user during arbitrary desk work. The concentration state evaluation device 1 has a pulse wave sensor 2, a signal processing unit 3, a display 4, an operation unit 5, a memory 6, and a processor 7.

The pulse wave sensor 2 is a sensor that detects pulse waves of a user, and for example, a photoelectric sensor including a light receiving unit and a light emitting unit. In the photoelectric sensor, light emitted from the light emitting unit is cast onto a living body (for example, the user's wrist), and the light receiving unit receives the reflected light or transmitted light thereof and outputs a signal corresponding to the amount of light received. Since the amount of light reflected or absorbed by the living body changes according to the blood flow rate in a blood vessel, the magnitude of the signal outputted from the photoelectric sensor increases or decreases according to the pulsation. Thus, the pulse wave sensor 2 outputs a pulse wave signal whose signal value increases or decreases in time series. However, the pulse wave sensor 2 is not limited to a photoelectric sensor and may be other sensors such as an electrocardiograph machine and an ultrasonic sensor.

The signal processing unit 3 has a sampling circuit that samples the pulse wave signal outputted from the pulse wave sensor 2, an amplification circuit that amplifies the pulse wave signal, and an A-D conversion circuit that converts the pulse wave signal to a digital signal, or the like. The signal processing unit 3 performs signal processing on the pulse wave signal by these circuits and inputs the signal-processed pulse wave signal to the processor 7.

The display 4 is a display unit that displays various information under the control of the processor 7.

The operation unit 5 accepts an input operation from the user. The operation unit 5 may be, for example, a button or the like, or may be a touch panel integrated with the display 4.

In this embodiment, a configuration where the display 4 and the operation unit 5 are provided in the concentration state evaluation device 1 is described as an example. However, such a configuration is not limiting. For example, an external device communicably coupled to the concentration state evaluation device 1 may have the display 4 and the operation unit 5. As the external device, for example, a portable terminal device such as a smartphone, a personal computer, or the like, can be employed as an example.

The memory 6 is a recording medium in which various programs including an evaluation program to evaluate the user concentration state and various data used for the various programs are recorded.

The processor 7 is, for example, an MPU (micro-processing unit), a CPU (central processing unit), or a DSP (digital signal processor) or the like. The processor 7 reads and executes various programs stored in the memory 6 and thus executes various computational processing. The processor 7 reads and executes a concentration state evaluation program recorded in the memory 6 and thus functions as a measurement unit 72, an index value calculation unit 73, and an evaluation unit 74. However, a part or all of the functions of the processor 7 may be implemented by dedicated hardware such as an ASIC (application-specific integrated circuit).

A signal acquisition unit 71 outputs a control command to the pulse wave sensor 2 and acquires a pulse wave signal from the pulse wave sensor 2 via the signal processing unit 3. The signal acquisition unit 71 stores the acquired pulse wave signal as pulse wave information (for example, reference pulse wave information or target pulse wave information, described later) in the memory 6. The pulse wave information is information including a pulse wave of the user, including a change in the signal value of the pulse wave signal in time series.

The measurement unit 72 acquires the pulse wave information from the memory 6, analyzes the pulse wave information, and measures the pulse rate and the interbeat interval (RRI or R-R interval) of pulses. The unit of the pulse rate and RRI is, for example, ms.

The index value calculation unit 73 calculates rMSSD (root mean square of successive differences) as the index value of the concentration state, based on the interbeat interval measured by the measurement unit 72. In this case, rMSSD is the root mean square of the difference between adjacent interbeat intervals for each predetermined time and is a value indicating the degree of change in the interbeat interval of pulses for each predetermined time. The predetermined time is a sufficiently shorter unit time (for example, three minutes) than the time of each of a resting period, a specified working period, and a desk work period, described later.

For example, the index value calculation unit 73 calculates rMSSD by the following equation (1):

$$rMSSD = \sqrt{\frac{1}{N - 1} {\sum }_{i = 1}^{N - 1} {( {x}_{i} - {x}_{i + 1} )}^{2}} . \qquad ( 1 )$$

In the equation (1), N is the number of interbeat intervals in the foregoing predetermined period and xi is an i-th interbeat interval in the predetermined period.

A data sorting unit 742 eliminates an index value (noise value) that can be an abnormal value, from the index value group calculated by the index value calculation unit 73, and thus sorts data. The data sorting unit 742 will be specifically described in detail later.

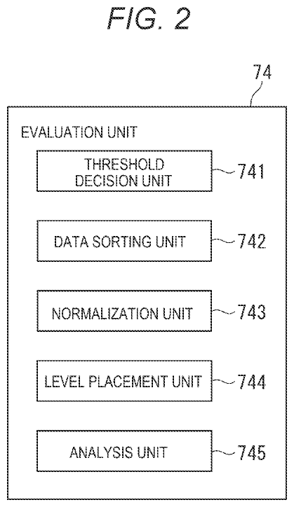

The evaluation unit 74 analyzes the index value group corresponding to the desk work period with reference to each of the index value group corresponding to the resting period and the index value group corresponding to the specified working period and thus evaluates the concentration state of the user during the desk work period. The evaluation unit 74 includes a threshold decision unit 741, the data sorting unit 742, a normalization unit 743, a level placement unit 744, and an analysis unit 745, as shown in FIG. 2. These units will be described in detail later.

### Concentration State Evaluation Method

A concentration state evaluation method according to this embodiment will now be described. The concentration state evaluation method according to this embodiment is a method of evaluating the concentration state of the user during the desk work period, using the foregoing concentration state evaluation device 1. In this method, a resting period, a specified working period, and a desk work period are set as described below.

The resting period is a period during which the user in the state of being awake (non-sleeping state) is at rest without carrying out any work.

The specified working period is a period during which the user carries out specified routine work. The specified working period in this embodiment includes a first working period and a second working period as a plurality of working periods with different types of work from each other. For example, during the first working period, an arbitrary mental arithmetic test is carried out as routine work that promotes convergent thinking of the user. During the second working period, an arbitrary creativity test is carried out as routine work that promotes divergent thinking of the user.

The desk work period is a period during which the user carries out arbitrary desk work. The type of the desk work is not particularly limited and may be, for example, the daily work of the user. Also, the desk work period may include a break time during the work and may include a plurality of periods with different contents of work from each other.

In this embodiment, the user spends the resting period (first time), the first working period, the resting period (second time), the second working period, and the desk work period, in this order. The resting periods (first time, second time), the first working period, and the second working period may preferably have a predetermined time. The desk work period may have a predetermined time or may have a time decided by a start operation and an end operation by the user.

In the description below, the period from the start of the resting period to the end of the second working period may be collectively referred to as a reference period.

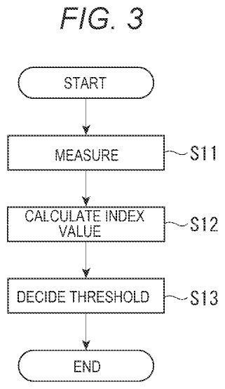

First, the concentration state evaluation device 1 executes the flowchart of FIG. 3. The user is assumed to wear the concentration state evaluation device 1 on the user's own living body part (for example, the user's wrist) before the start of the flowchart of FIG. 3.

In the flowchart of FIG. 3, first, while the user spends the reference period, the signal acquisition unit 71 acquires a pulse wave signal from the pulse wave sensor 2 via the signal processing unit 3 and stores the pulse wave signal as reference pulse wave information in the memory 6. Then, the measurement unit 72 acquires the reference pulse wave information, analyzes the reference pulse wave information, and thus measures the interbeat interval (RRI) (step S11). The reference pulse wave information includes pulse wave information corresponding to each period in the reference period, that is, the resting period (first time), the first working period, the resting period (second time), and the second working period. The measurement unit 72 can measure the interbeat interval in each period.

In step S11, the user may carry out a start operation to start each period in the reference period via the operation unit 5. In this case, the signal acquisition unit 71 can acquire a pulse wave signal corresponding to each period in the reference period and store the pulse wave signal as reference pulse wave information in the memory 6, in response to the start operation by the user.

Alternatively, in step S11, the user may carry out a start operation to start the reference period via the operation unit 5, and with the lapse of the time of each period in the reference period that is predetermined, a transition of each period may take place. In this case, the signal acquisition unit 71 can acquire pulse wave information corresponding to each period according to the lapse of the time of each period in the reference period that is predetermined, after the start operation by the user. In this case, the processor 7 may guide the user about the start and end of each period via the display 4 or an audio unit, not illustrated.

Next, the index value calculation unit 73 calculates an index value (that is, rMSSD) in each period in the reference period, based on the interbeat interval measured in step S11 (step S12). The index value calculation unit 73 can find an index value group corresponding to the resting period (for example, the total the first time and the second time or one of the first time and the second time), an index value group corresponding to the first working period, and an index value group corresponding to the second working period.

Next, the threshold decision unit 741 calculates working thresholds Vt1, Vt2 and a resting threshold Vt3 for the index value (step S13).

Specifically, the threshold decision unit 741 eliminates an index value corresponding to a predetermined elimination period including the boundary between adjacent periods in the reference period, from the entire index value groups calculated in step S12. The elimination period may be preferably a predetermined time or longer at the time of calculating the index value.

Subsequently, the median value of the index value group corresponding to the first working period is calculated as the working threshold Vt1. The median value of the index value group corresponding to the second working period is calculated as the working threshold Vt2. The median value of the index value group corresponding to the resting period is calculated as the resting threshold Vt3.

Normally, the magnitudes of these thresholds are in the relationship of the working thresholds Vt1, Vt2<the resting threshold Vt3. In this embodiment, the working threshold Vt1<the working threshold Vt2. However, the relationship of the magnitudes of the working thresholds Vt1, Vt2 may be reversed. Each of the working thresholds Vt1, Vt2 and the resting threshold Vt3 is not limited to the median value of the index value group corresponding to each period and may be found by other calculation methods.

Hereinafter, the working thresholds Vt1, Vt2 and the resting threshold Vt3 may be simply referred to as the thresholds Vt1 to Vt3.

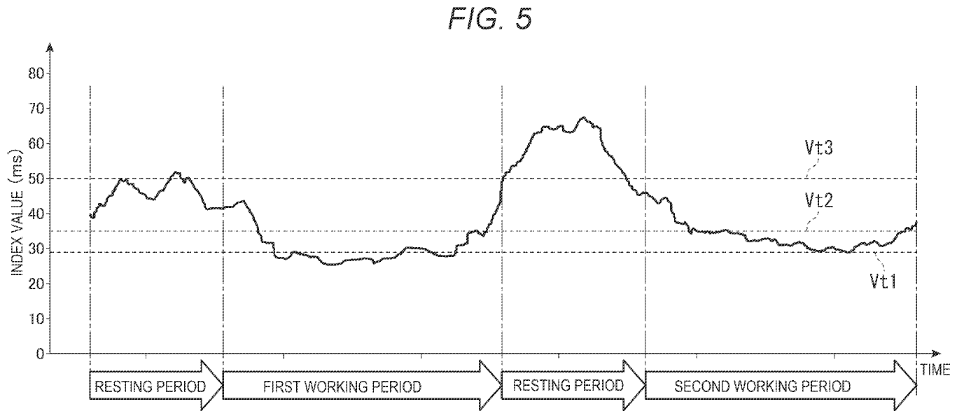

The flowchart of FIG. 3 thus ends. As a result of the processing shown in the flowchart of FIG. 3, data as shown in the graph of FIG. 5 is acquired. FIG. 5 shows a transition of the index value in the reference period and the thresholds Vt1 to Vt3 calculated from the index value group corresponding to each period in the reference period.

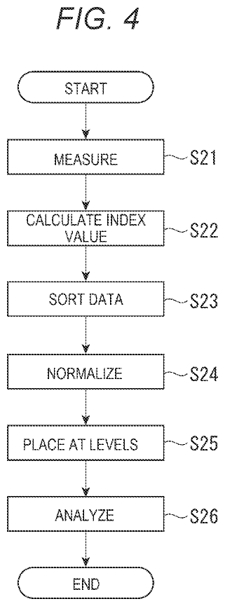

Next, the concentration state evaluation device 1 executes the flowchart of FIG. 4. The flowchart of FIG. 4 may be started immediately after the end of the foregoing step S13 or may be started after the lapse of an arbitrary interval period after the end of the foregoing step S13.

In the flowchart of FIG. 4, first, while the user spends the desk work period, the signal acquisition unit 71 acquires a pulse wave signal from the pulse wave sensor 2 via the signal processing unit 3 and stores the pulse wave signal as target pulse wave information in the memory 6. Then, the measurement unit 72 analyzes the target pulse wave information and thus measures the pulse rate and the interbeat interval (RRI) (step S21). The target pulse wave information includes pulse wave information corresponding to the desk work period. The measurement unit 72 can measure the interbeat interval in the desk work period.

In step S21, the user may carry out a start operation and an end operation for the desk work period via the operation unit 5. In this case, the signal acquisition unit 71 can acquire pulse wave information corresponding to the desk work period in response to the operation by the user to the operation unit 5.

Alternatively, in step S21, the user may carry out a start operation for the desk work period via the operation unit 5, and the desk work period may end with the lapse of a predetermined time. In this case, the signal acquisition unit 71 can acquire pulse wave information corresponding to the desk work period according to the lapse of the time of the desk work period that is predetermined, after the start operation by the user. In this case, the processor 7 may guide the user about the end of the desk work period via the display 4 or an audio unit, not illustrated.

Next, the index value calculation unit 73 calculates an index value (that is, rMSSD) in the desk work period, based on the interbeat interval measured in step S21 (step S22). The index value calculation unit 73 can thus find an index value group corresponding to the desk work period.

Step S22 may be started before the end of step S21. In this case, the index value calculation unit 73 may calculate an index value every time the interbeat interval of the predetermined time is measured in step S21.

Next, the data sorting unit 742 eliminates an index value (noise value) that can be an abnormal value, from the index value group calculated in step S22 (step S23).

The data sorting unit 742 can specify the noise value by at least one method or a combination of a plurality of methods of methods (1) to (6) given below.
(1) Based on the pulse rate measured in step S21, a period during which the pulse rate is 120 bpm or higher or 54 bpm or lower (pulse rate abnormality period) is specified and an index value corresponding to the pulse rate abnormality period is specified as the noise value.
(2) Based on the interbeat interval measured in step S21, a period during which the interbeat interval is 400 ms or shorter or 1500 ms or longer (interbeat interval abnormality period) is specified and an index value corresponding to the interbeat interval abnormality period is specified as the noise value.
(3) Data formed of the index values calculated in step S22 and rearranged in descending order is created and this data is equally divided (for example, equally divided into ten parts) to create a plurality of groups. Index values belonging to the first group and the last group are specified as the noise value.
(4) The desk work period is divided into a plurality of sections. An average m and a standard deviation σ in each section with respect to the index value group calculated in step S22 are found. An index value that is out of a range of 2σ in each section is specified as the noise value.
(5) An absolute value K1 of allowable error of the index value is set. When each index value calculated in step S22 has a difference of K1 or more from the average of the entire index value group calculated in step S22, this index value is specified as the noise value.
(6) An absolute value K2 of allowable error of the index value is set. When each index value calculated in step S22 has a difference of K2 or more from the adjacent index value, this index value is specified as the noise value.

In the methods (1) and (2), the threshold of the pulse rate to specify the pulse rate abnormality period and the threshold of the interbeat interval to specify the interbeat interval abnormality period can be set arbitrarily. Each of the methods (5) and (6) may be executed a plurality of times, depending on the noise level of the index value group calculated in step S22.

Next, the normalization unit 743 normalizes the index value group sorted in step S23 (step S24).

Specifically, the normalization unit 743 decides a minimum value Vmin and a maximum value Vmax of index values from an arbitrary range including the index value group calculated at least in one of steps S12 and S22. Thus, an analysis range (a range of the minimum value Vmin or higher to the maximum value Vmax or lower) for the index value group corresponding to the desk work period is decided. In this example, Vmin<Vt1, Vt2, and Vt3<Vmax.

The normalization unit 743 divides the analysis range into a plurality of sections, based on the thresholds Vt1 to Vt3 calculated in step S13, and normalizes, for each section, the index value group sorted in step S23 to a range corresponding to the section. In this embodiment, the individual sections correspond to division ranges formed by dividing a range of 0 to 1 and the entirety of the analysis range corresponds to the range of 0 to 1.

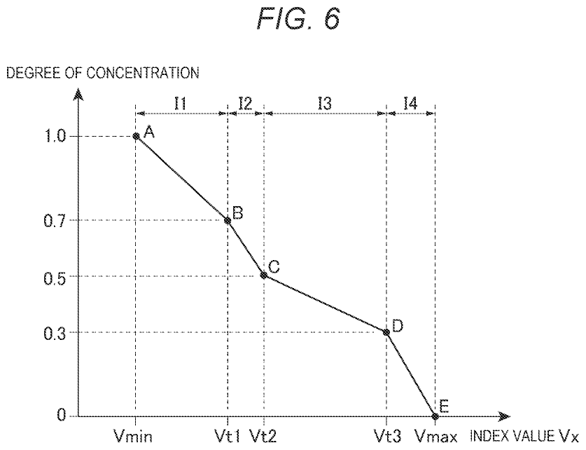

An example of the normalization by the normalization unit 743 will now be described with reference to the graph of FIG. 6.

As shown in FIG. 6, the normalization unit 743 divides an evaluation range for an index value Vx into a range of the minimum value Vmin<the index value Vx≤the threshold Vt1 (section I1), a range of the threshold Vt1<the index value Vx≤the threshold Vt2 (section I2), a range of the threshold Vt2<the index value Vx≤the threshold Vt3 (section I3), and a range of the threshold Vt3<the index value Vx≤the maximum value Vmax (section I4).

The normalization unit 743 then normalizes the index value group sorted in step S23 to a division range corresponding to each of the sections I1 to I4.

For example, in the range of the minimum value Vmin<the index value Vx≤the threshold Vt1 (section I1), the index value Vx is normalized to a division range from 1 to 0.7 by the equation of a straight line passing through a point A (Vmin, 1) and a point B (Vt1, 0.7).

In the range of the threshold Vt1<the index value Vx≤the threshold Vt2 (section I2), the index value Vx is normalized to a division range from 0.7 to 0.5 by the equation of a straight line passing through the point B (Vt1, 0.7) and a point C (Vt2, 0.5).

In the range of the threshold Vt2<the index value Vx≤the threshold Vt3 (section I3), the index value Vx is normalized to a division range from 0.5 to 0.3 by the equation of a straight line passing through the point C (Vt2, 0.5) and a point D (Vt3, 0.3).

In the range of the threshold Vt3<the index value Vx≤the maximum value Vmax (section I4), the index value Vx is normalized to a division range from 0.3 to 0 by the equation of a straight line passing through the point D (Vt3, 0.3) and a point E (Vmax, 0).

When the index value Vx≤the minimum value Vmin, the index value Vx is converted to 1. When the maximum value Vmax<the index value Vx, the index value Vx is converted to 0.

The index value normalized to the range of 0 to 1 is thus found. Hereinafter, the normalized index value may be referred to as the degree of concentration.

In the example shown in FIG. 6, the value to which the threshold Vt1 is normalized (normalization threshold Ct1) is 0.7, the value to which the threshold Vt2 is normalized (normalization threshold Ct2) is 0.5, and the value to which the threshold Vt3 is normalized (normalization threshold Ct3) is 0.3. However, these values Ct1 to Ct3 can be arbitrarily set.

The range to which the index value group is normalized is not limited to the range of 0 to 1 and may be any range. In this embodiment, the relationship of the magnitude of values is reversed when converting the index value to the degree of concentration. However, this is not limiting.

Next, the level placement unit 744 places the threshold group normalized in step S24, that is, the degree-of-concentration group, at a plurality of levels of concentration, that is, levels L1 to L4 (step S25). As criteria for the level placement, the normalization thresholds Ct1 to Ct3 are used. For example, for a degree of concentration Cx during the desk work period, the level placement unit 744 determines that it is the level L1 when the degree of concentration Cx≤the threshold Ct3. The level placement unit 744 determines that it is the level L2 when the threshold Ct3<the degree of concentration Cx≤the threshold Ct2. The level placement unit 744 determines that it is the level L3 when the threshold Ct2<the degree of concentration Cx≤the threshold Ct1. The level placement unit 744 determines that the level L4 when the threshold Ct1<degree of concentration Cx.

Next, the analysis unit 745 analyzes the degree-of-concentration group placed at the levels in step S25 (step S26).

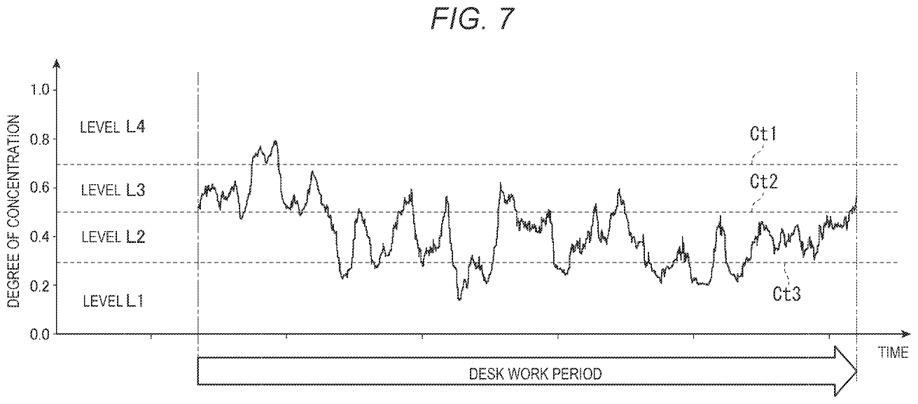

For example, the analysis unit 745 may generate a graph as illustrated in FIG. 7, as result information representing a change with time across the plurality of levels of concentration (levels L1 to L4) during the desk work period. FIG. 7 shows the change with time across the levels L1 to L4, based on the transition of the degree of concentration during the desk work period and the ranges of the levels L1 to L4 of the degree of concentration. When a task name (meeting, telework, break or the like) is stored corresponding to each time bracket in the desk work period, a graph in which the time bracket corresponding to each task is demarcated may be shown.

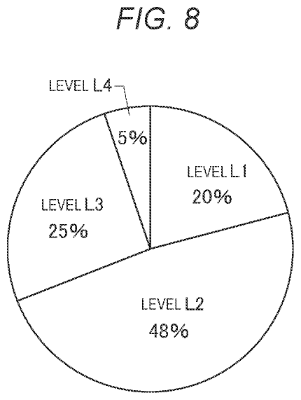

The analysis unit 745 may also generate a pie chart as illustrated in FIG. 8, as result information representing the time proportion of each of the levels of concentration (levels L1 to L4) during the desk work period. FIG. 8 shows the time proportion of each of the levels L1 to L4 during the desk work period by percentage.

The flowchart of FIG. 4 thus ends and the concentration state evaluation method according to this embodiment ends accordingly. The processor 7 may cause the display 4 to display the various result information generated in step S26 or may transmit the various result information to another information processing device, in response to a request inputted to the operation unit 5.

In this embodiment, the processor 7 can store, in the memory 6, the data acquired or calculated in each of the foregoing steps, and can refer to the data according to need. The processor 7 may store, in the memory 6, the data acquired or calculated in each of the foregoing steps in association with the identification information of the user (user ID).

For example, when executing the concentration state evaluation method according to this embodiment for an arbitrary user A, the processor 7 acquires the user ID of the user A and determines whether data associated with this user ID is stored in the memory 6 or not. When the thresholds Vt1 to Vt3 for the user A are already stored in the memory 6, the execution of the flowchart of FIG. 3 may be omitted.

### Advantageous Effects of This Embodiment

The concentration state evaluation method according to this embodiment is a method for the processor 7 as a computer to evaluate the concentration state of a user. The processor 7 functions as the measurement unit 72, the index value calculation unit 73, and the evaluation unit 74, as described above. The measurement unit 72 executes: the first measurement step (step S11) of acquiring reference pulse wave information that represents a pulse wave during each period of a resting period and a specified working period of the user, and measuring an interbeat interval during each period of the resting period and the specified working period, based on the reference pulse wave information; and the second measurement step (step S21) of acquiring target pulse wave information that represents a pulse wave during an arbitrary desk work period of the user, and measuring an interbeat interval during the desk work period, based on the target pulse wave information. The index value calculation unit 73 executes the index value calculation step (steps S12, S22) of calculating rMSSD, which is a root mean square of a difference between adjacent interbeat intervals for each predetermined time, as an index value of the concentration state, based on the interbeat interval measured in the first measurement step or the second measurement step, and finding an index value group corresponding to each period of the resting period, the specified working period, and the desk work period. The evaluation unit 74 executes the evaluation step (steps S13, S23 to S26) of analyzing the index value group corresponding to the desk work period with reference to each of the index value group corresponding to the resting period and the index value group corresponding to the specified working period, and thus evaluating the concentration state of the user during the desk work period.

With such a method, the index value group corresponding to the resting period, which is the reference information for the state where the concentration of the user is low, and the index value group corresponding to the specified working period, which is the reference information for the state where the concentration of the user is high, can be acquired as the personal biometric information of the user. As the index value group corresponding to the desk work period is evaluated with reference to the index value groups corresponding to the resting period and the specified working period, the concentration state of the user during the desk work period can be accurately evaluated.

Generally, rMSSD is strongly related to parasympathetic nerves. In this embodiment, rMSSD of the same user tends to greatly differ between the specified working period, during which the user is required to concentrate, and the resting period, during which the user is required to rest. Therefore, rMSSD can be used as the index value of the concentration state of the user.

In this embodiment, the evaluation unit 74 includes the threshold decision unit 741 and the level placement unit 744. The threshold decision unit 741 executes the threshold decision step (step S13) of deciding the resting threshold Vt3 based on the index value group corresponding to the resting period, and deciding the working thresholds Vt1, Vt2 based on the index value group corresponding to the specified working period. The level placement unit 744 executes the level placement step (step S25) of placing the index value group corresponding to the desk work period at a plurality of levels of concentration, based on the thresholds Vt1 to Vt3.

With such a method, the concentration state of the user during the desk work period can be suitably evaluated.

In this embodiment, the evaluation unit 74 further includes the normalization unit 743. The normalization unit 743 executes the normalization step (step S24) of dividing the analysis range of the index value group corresponding to the desk work period into the plurality of sections I1 to I4, based on the thresholds Vt1 to Vt3, and normalizing, for each of the sections, the index value group corresponding to the desk work period to a range corresponding to the section, before the level placement step (step S25).

With such a method, the concentration state of the user during the desk work period can be evaluated more accurately.

In this embodiment, the specified working period includes a plurality of working periods with different types of work from each other. The threshold decision unit 741 decides the thresholds Vt1, Vt2 corresponding to the working periods, respectively.

With such a method, the concentration state of the user during the desk work period can be evaluated more accurately.

In this embodiment, the measurement unit 72 measures each of an interbeat interval and a pulse rate during the desk work period, based on the target pulse wave information, in the second measurement step (step S21). The evaluation unit 74 further includes the data sorting unit 742. The data sorting unit 742 executes the data sorting step (step S23) of specifying a pulse rate abnormality period in which the pulse rate is out of a predetermined range during the desk work period, and eliminating the index value corresponding to the pulse rate abnormality period from the index value group corresponding to the desk work period, before the level placement step (step S25).

With such a method, a heart rate unsuitable for the evaluation, such as a high heart rate during exercise or a lower heart rate than the normal heart rate when the user is awake, can be eliminated. Therefore, the concentration state during the desk work can be evaluated more accurately.

In this embodiment, the measurement unit 72 further includes the analysis unit 745. The analysis unit 745 may generate result information representing the change with time across the plurality of levels of concentration during the desk work period, in the analysis step (step S26). The analysis unit 745 may generate result information representing the time proportion of each of the levels of concentration during the desk work period, in the analysis step (step S26).

With such a method, the generated result information can be presented to the user via the display 4 or the like and thus can enable the user to easily grasp the user's own concentration state.

### MODIFICATION EXAMPLES

The present disclosure is not limited to the foregoing embodiment. Any configuration acquired by suitably combining modifications, improvements, and the embodiment within a range that can achieve the objective of the present disclosure, or the like, is included in the present disclosure.

### Modification Example 1

In the foregoing embodiment, the specified working period includes the first working period and the second working period. However, this is not limiting. For example, the specified working period may be one working period or may include three or more working periods. In this case, the threshold decision unit 741 may calculate a working threshold for each working period and thus decide one or three or more working thresholds, in the threshold decision step (step S13). Consequently, the degree of concentration may be placed at three or five or more levels.

### Modification Example 2

In the execution of the concentration state evaluation method according to the foregoing embodiment, the user spends the resting period (first time), the first working period, the resting period (second time), the second working period, and the desk work period in this order. However, the order of these periods is not limited to this.

For example, when the user performs an operation to select one of the periods via the operation unit 5, the signal acquisition unit 71 can determine that a selection period is started in response to an input of operation information, and can acquire a pulse wave signal corresponding to the selection period.

The number of resting periods is not limited to two and may be one or three or more.

### Modification Example 3

In the foregoing embodiment, the measurement unit 72 measures not only the interbeat interval but also the pulse rate. However, when the pulse rate is not used (for example, when the specification of the noise value using the pulse rate is not executed), the measurement of the pulse rate may be omitted.

### Modification Example 4

In the foregoing embodiment, pulse wave information corresponding to another period than the reference period and the desk work period may be acquired as well, and an index value group may be calculated based on the pulse wave information. For example, the normalization unit 743 may decide the minimum value Vmin and the maximum value Vmax from an index value group corresponding to the user's awake time bracket of a day, and may decide the analysis range of the index value group for the user.

### Modification Example 5

In the foregoing embodiment, the data sorting unit 742 may perform processing to eliminate the noise value, targeting not only the index value group corresponding to the desk work period but also the index value group corresponding to the reference period. Specifically, in the foregoing embodiment, the data sorting unit 742 may perform the step of eliminating the noise value from the index value group corresponding to the reference period before the threshold decision step (step S13).

### Modification Example 6

In the foregoing embodiment, the flowchart of FIG. 4 is executed after the flowchart of FIG. 3 is executed. However, this is not limiting. For example, the index value calculation steps (steps S12 and S22) may be executed together after the first measurement step (step S11) and the second measurement step (step S12) are executed. The order of the first measurement step (step S11) and the second measurement step (step S12) may be reversed.

### Modification Example 7

In the evaluation step in the foregoing embodiment, the evaluation unit 74 functions as the threshold decision unit 741, the data sorting unit 742, the normalization unit 743, and the level placement unit 744. However, this is not limiting. That is, while the evaluation step includes the threshold decision step (step S13), the data sorting step (step S23), the normalization step (step S24), and the level placement step (step S25), this is not limiting.

For example, in the foregoing embodiment, when the data sorting step (step S23) is omitted, all the index value groups calculated in step S22 may be used.

Also, in the foregoing embodiment, when the normalization step (step S24) is omitted, the index value group that is not normalized may be placed at an appropriate level, based on the thresholds Vt1 to Vt3.

Also, in the foregoing embodiment, when the threshold decision step (step S13) and the level placement step (step S25) are omitted, the evaluation unit 74 may analyze the index value group corresponding to the desk work period, by any other technique. For example, the evaluation unit 74 may find an overlap range where a transition range of the index value corresponding to the desk work period overlaps each of a transition range of the index value corresponding to the resting period and a transition range of the index value corresponding to the specified working period, and may analyze the index value group corresponding to the desk work period, based on the ratio of the resulting two overlap ranges. In such a case, the evaluation unit 74 may evaluate the overall concentration state during the desk work period.

### Modification Example 8

The concentration state evaluation device 1 according to the foregoing embodiment is configured as a wearable device configured to be wearable on a human body. However, this is not limiting.

For example, the concentration state evaluation device 1 may be configured as an information processing device such as a smartphone or a personal computer and may not have the pulse wave sensor 2 and the signal processing unit 3. In this case, the concentration state evaluation device 1 may be configured to be communicable with a wearable device having the pulse wave sensor 2 and the signal processing unit 3 and may acquire pulse wave information from the wearable device. Also, when pulse wave information of each user is stored in an external storage device such as s server device, the concentration state evaluation device 1 may acquire pulse wave information corresponding to an evaluation target user from the external storage device.

### Modification Example 9

In the concentration state evaluation device 1 according to the foregoing embodiment, the one processor 7 is equivalent to the computer according to the present disclosure. However, the computer according to the present disclosure may be formed by a plurality of processors.

For example, a concentration state evaluation device according to a modification example may be formed by a wearable device and an information processing device such as a smartphone or a personal computer. The concentration state evaluation method according to this modification example may be executed by the processor of the wearable device and the processor of the information processing device. In this case, the processor of the wearable device may execute the first measurement step (step S11) and the second measurement step (step S21) and may further execute the index value calculation step (steps S12, S22). The processor of the information processing device may execute the rest of the steps.

What is claimed is:

1. A concentration state evaluation method for a computer to evaluate a concentration state of a user, the concentration state evaluation method causing the computer to execute:
a first measurement step of acquiring reference pulse wave information that represents a pulse wave detected by a pulse wave sensor worn by the user and that represents a pulse wave during each period of a resting period and a specified working period of the user, and measuring an interbeat interval during each period of the resting period and the specified working period, based on the reference pulse wave information;
a second measurement step of acquiring target pulse wave information that represents a pulse wave detected by the pulse wave sensor and that represents a pulse wave during an arbitrary desk work period of the user, and measuring an interbeat interval during the desk work period, based on the target pulse wave information;
an index value calculation step of calculating rMSSD, which is a root mean square of a difference between adjacent interbeat intervals for each predetermined time, as an index value of the concentration state, based on the interbeat interval measured in the first measurement step or the second measurement step, and finding an index value group corresponding to each period of the resting period, the specified working period, and the desk work period; and
an evaluation step of analyzing the index value group corresponding to the desk work period with reference to each of the index value group corresponding to the resting period and the index value group corresponding to the specified working period, and thus evaluating the concentration state of the user during the desk work period.

2. The concentration state evaluation method according to claim 1, wherein
the evaluation step includes:
a threshold decision step of deciding a resting threshold based on the index value group corresponding to the resting period, and deciding a working threshold based on the index value group corresponding to the specified working period; and
a level placement step of placing the index value group corresponding to the desk work period at a plurality of levels of concentration, based on the resting threshold and the working threshold.

3. The concentration state evaluation method according to claim 2, wherein
the evaluation step further includes:
a normalization step of dividing an analysis range of the index value group corresponding to the desk work period into a plurality of sections, based on the working threshold and the resting threshold, and normalizing, for each of the sections, the index value group corresponding to the desk work period to a range corresponding to the section, before the level placement step.

4. The concentration state evaluation method according to claim 2, wherein
the specified working period includes a plurality of working periods with different types of work from each other, and
the threshold decision step decides a plurality of thresholds corresponding to the periods, working respectively, as the working threshold.

5. The concentration state evaluation method according to claim 2, wherein
the second measurement step measures each of an interbeat interval and a pulse rate during the desk work period, based on the target pulse wave information, and
the evaluation step further includes
a data sorting step of specifying a pulse rate abnormality period in which the pulse rate is out of a predetermined range during the desk work period, and eliminating the index value corresponding to the pulse rate abnormality period from the index value group corresponding to the desk work period, before the level placement step.

6. The concentration state evaluation method according to claim 2, wherein
the evaluation step further includes an analysis step of generating result information representing a change with time across the plurality of levels of concentration during the desk work period, after the level placement step.

7. The concentration state evaluation method according to claim 2, wherein
the evaluation step further includes an analysis step of generating result information representing a time proportion of each of the levels of concentration during the desk work period, after the level placement step.

8. A non-transitory computer-readable storage medium storing a concentration state evaluation program that is readable and executable by a computer,
the concentration sate evaluation program causing the computer to execute the concentration state evaluation method according to claim 1.

9. A concentration state evaluation device comprising:
a measurement unit that acquires pulse wave information representing a pulse wave detected by a pulse wave sensor worn by a user and representing a pulse wave during each period of a resting period, a specified working period, and an arbitrary desk work period of the user, and that measures an interbeat interval during each of the periods, based on the pulse wave information;
an index value calculation unit that calculates rMSSD, which is a root mean square of a difference between adjacent interbeat intervals for each predetermined time, as an index value of a concentration state, based on the interbeat interval measured by the measurement unit, and that finds an index value group corresponding to each period of the resting period, the specified working period, and the desk work period; and
an evaluation unit that analyzes the index value group corresponding to the desk work period with reference to each of the index value group corresponding to the resting period and the index value group corresponding to the specified working period, and thus evaluates the concentration state of the user during the desk work period.
